(19) United States
(12) Patent Application Publication
(10) Pub. No.: US 2021/0395078 A1
(43) Pub. Date: Dec. 23, 2021

(54) Preparation Method Of Miniature Solid Silicon Needle

(71) Applicant: Zhu Hongfei, Suzhou (CN)

(72) Inventor: Zhu Hongfei, Suzhou (CN)

(21) Appl. No.: 17/445,455

(22) Filed: Aug. 19, 2021

(30) Foreign Application Priority Data
Jun. 18, 2020 (CN) CN111675190A

(51) Int. Cl. B81C 1/00; C23C 16/34; C23C 16/50; C23C 16/56; C23C 16/02; G03F 7/16; G03F 7/26

(52) U.S. Cl. CPC B81C 1/00111; C23C 16/345; C23C 16/50; C23C 16/56; C23C 16/0272; A61M 2037/0053; G03F 7/26; B81C 2201/0112; B81C 2201/0133; B81C 2201/0178; B81C 2201/0132; G03F 7/162

(57) ABSTRACT

The present invention, in some embodiments thereof, provides a preparation method of a miniature solid silicon needle. The preparation method includes the following steps: growing one layer of silicon dioxide on a surface of monocrystalline silicon; depositing one layer of silicon nitride protective film on a surface of the silicon dioxide; coating a surface of the silicon nitride protective film with photoresist; and performing exposing, developing and etching, wherein the protective film adopts silicon nitride and is capable of accelerating etching reaction in the process of etching silicon, so that a diameter of a base of the silicon needle is smaller. According to the present invention, the process is simple, and the solid silicon needle has high durability and is suitable for transdermal drug permeation of biomacromolecule drugs.

### TECHNICAL FIELD

The present invention relates to the technical field of surface micro-processing and manufacturing of a semiconductor silicon material, and more particularly, to a preparation method of a miniature solid silicon needle.

### BACKGROUND OF THE INVENTION

Due to the degradation of drugs in stomach and intestine and the first-pass effect of the liver, most of the oral drugs have mostly or completely failed before arriving at the site of action. In addition, the drug adaptability of patients is also a problem, and most of oral medication needs medication at a certain intervals during treatment, which brings many inconveniences to patients.

In addition, another common administration mode is injection which can make drugs penetrate through a biological barrier, including subcutaneous injection and intravenous infusion. Although this method is effective, injection usually brings additional pain to patients and is liable to cause local skin injury and bleeding at the injection point, thereby increasing the risk of disease infection.

There is a novel administration technology, that is, transdermal administration. Scalp administration refers to a dosage form that is administrated on the surface of the skin, so that the drug passes through all layers of the skin at a nearly constant speed and is absorbed by capillaries to enter circulation to produce a systemic or local therapeutic effect.

Chinese invention patent CN1569271A discloses a miniature solid silicon needle array chip, and a preparation method and application thereof. In this patent, a metal film serves as a protective film, a diameter of a micro-needle tip of the prepared miniature solid silicon needle is 10 nm to 10 μm, and a diameter of the bottom of the micro-needle is 20-300 μm. It is well known that while the breaking rate of the micro-needles is controlled, the smaller the diameter of the micro-needle body is, the smaller the wound of human skin is, and the easier the micro-needle is to penetrate the skin.

Therefore, for the above problems, a preparation method of a miniature solid silicon needle is provided so as to prepare the miniature solid silicon needle which has a thinner needle body, is more convenient to penetrate through the skin and causes a smaller wound.

### SUMMARY OF THE INVENTION

The following summary is an explanation of some of the general inventive steps for the system, method, architecture and apparatus in the description. This summary is not an extensive overview of the invention and does not intend to limit the scope beyond what is described and claimed as a summary.

In order to overcome the defects in the prior art, an objective of the present invention is to provide a preparation method of a miniature solid silicon needle.

In order to achieve the above objective and other related objectives, the present invention provides the following technical solution: a preparation method of a miniature solid silicon needle includes the following steps:

(1) selecting a single-side or double-side polished monocrystalline silicon wafer for cleaning and drying;

(2) growing one layer of silicon dioxide on the monocrystalline silicon wafer;

(3) depositing one layer of silicon nitride on the silicon dioxide;

(4) spin-coating the silicon nitride with photoresist, and transferring a pattern on a mask to the photoresist through a photoetching process, wherein the pattern on the mask is an array-shaped round spot, and the photoetched photoresist forms an array-shaped round shielding adhesive film;

(5) sequentially removing the silicon nitride located outside the shielding adhesive film and the silicon dioxide below the shielding adhesive film and exposing the monocrystalline silicon wafer;

(6) performing anisotropic etching on the exposed monocrystalline silicon wafer in the step 5 by utilizing an inductively coupled plasma etching system and adopting a deep silicon etching Bosch process, and etching an array-shaped cylinder on the monocrystalline silicon wafer;

(7) sequentially removing the shielding adhesive film, the silicon nitride and the silicon dioxide at the top end of the cylinder, and performing isotropic etching on the top end of the cylinder through KOH to form a solid micro-needlepoint cone needle and obtain a miniature solid silicon needle array chip; and

(8) coating a needle body surface of the miniature solid silicon needle array chip with the photoresist, transferring the pattern on the mask to the photoresist through a photoetching process, then performing etching to form a grid-shaped flow guide groove between the silicon needles, and then removing the shielding adhesive film, wherein the pattern of the mask is a checkerboard-shaped block and the photoetched photoresist forms a block-shaped shield adhesive film.

The preferred technical solution is as follows: in the step 1, the monocrystalline silicon wafer is N type, and has resistivity ranging from 1 to 10 Ω·cm and a thickness of 700±10 μm.

The preferred technical solution is as follows: in the step 2, one layer of silicon dioxide is oxidized to grow on the monocrystalline silicon wafer according to the order of wet oxygen and dry oxygen, the oxidizing temperature is controlled to below 1100° C., and the wet oxygen time and the dry oxygen time are both 5 hours.

The preferred technical solution is as follows: in the step 3, a substrate is deposited by PECVD at a temperature of 300-450° C. and a pressure intensity of 10-270 Pa.

The preferred technical solution is as follows: the substrate is deposited by PECVD at a temperature of 400° C.

The preferred technical solution is as follows: in the step 3, one layer of silicon nitride with a thickness of 200 nm is deposited on a surface of adopted dioxide by PECVD.

The preferred technical solution is as follows: in the step 5, the silicon nitride exposed outside the shielding adhesive film is subjected the cylinder through dry etching, the silicon dioxide is exposed, and the silicon dioxide is subjected to wet etching.

The preferred technical solution is as follows: in the step 5, the silicon nitride exposed outside the shielding adhesive film and the silicon dioxide located below the silicon nitride may be etched by an ion beam.

The preferred technical solution is as follows: the silicon nitride is subjected to dry etching with a reactive ion etching machine, and after the silicon dioxide is exposed, the silicon dioxide is subjected to wet etching with mixed liquid of hydrogen fluoride and hydrofluoric acid until the monocrystalline silicon wafer is exposed.

The preferred technical solution is as follows: in the step 7, a KOH solution of 40-80 degrees is used for etching, and the etching time is 30-70 minutes.

Due to the application of the above technical solutions, compared with the prior art, the present invention has the following advantages:

similarly, the exposed monocrystalline silicon wafer is subjected to anisotropic etching by utilizing an inductively coupled plasma etching system and adopting a deep silicon etching Bosch process, and an array-shaped cylinder is etched on the monocrystalline silicon wafer. In the background art, a metal layer serves as a protective layer, but in the present invention, the silicon nitride serves as the protective layer, the silicon nitride can accelerate the internal etching reaction in the deep silicon etching process, so that the cylinder is thinner, and the diameter of the finally obtained needle body is thinner.

### BRIEF DESCRIPTION OF FIGURES

The novel features believed to be characteristic of the illustrative embodiments are set forth in the appended claims. The illustrative embodiments, however, as well as a preferred mode of use, further objectives and descriptions thereof, will best be understood by reference to the following detailed description of one or more illustrative embodiments of the present disclosure when read in conjunction with the accompanying drawings, wherein:

FIG. 1 is a picture of a solid silicon needle prepared by a background technical solution.

FIG. 2 is a picture of a solid silicon needle supported by the technical solution of the present invention.

FIG. 3 is a schematic flow chart of the technology of the present invention.

FIG. 4 is a mask pattern in step 4 of the present invention.

FIG. 5 is a mask pattern in step 8 of the present invention.

### DETAILED DESCRIPTION

Hereinafter, the preferred embodiment of the present invention will be described in detail with reference to the accompanying drawings. The terminologies or words used in the description and the claims of the present invention should not be interpreted as being limited merely to their common and dictionary meanings. On the contrary, they should be interpreted based on the meanings and concepts of the invention in keeping with the scope of the invention based on the principle that the inventor(s) can appropriately define the terms in order to describe the invention in the best way.

It is to be understood that the form of the invention shown and described herein is to be taken as a preferred embodiment of the present invention, so it does not express fully the technical spirit and scope of this invention. Accordingly, it should be understood that various changes and modifications may be made to the invention without departing from the spirit and scope thereof.

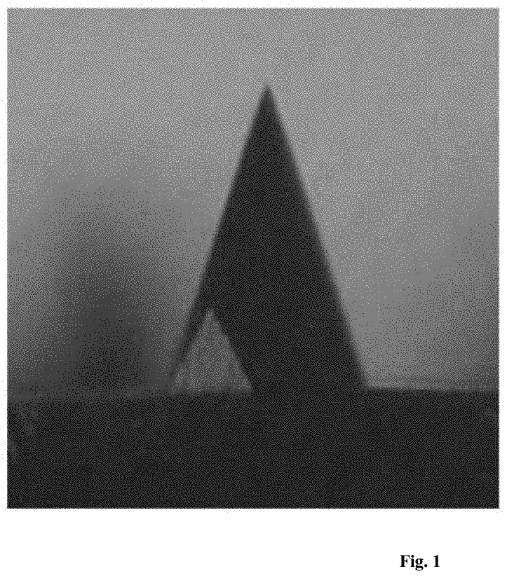

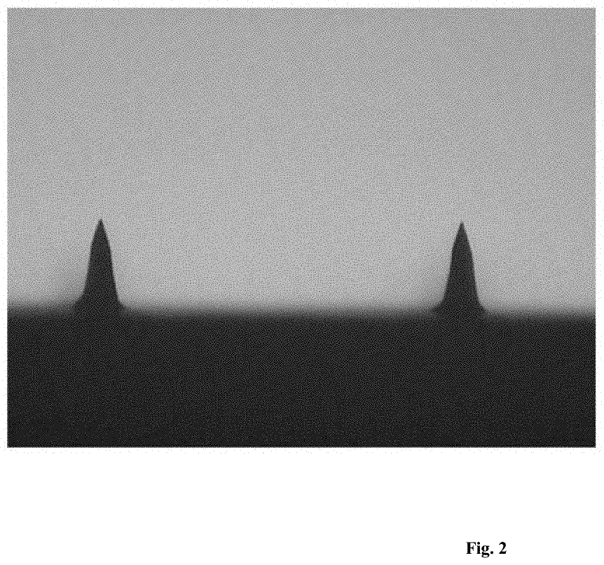

Refer to FIG. 1 to FIG. 5. In the description of the present invention, it should be noted that orientations or position relationships indicated by terms “center”, “upper”, “lower”, “left”, “right”, “vertical”, “horizontal”, “inner”, “outer”, etc. are orientation or position relationships shown in the accompanying drawings, and these terms are only used to facilitate description of the present invention and simplify the description, but not to indicate or imply that the mentioned apparatus or components must have a specific orientation or must be established and operated in a specific orientation, and thus these terms cannot be understood as a limitation to the present invention. In addition, the terms such as “first”, “second”, and “third” are used only for the purpose of description and cannot be understood to indicate or imply relative importance.

Terms such as “horizontal”, “vertical” and “overhanging” do not mean that a component is absolutely horizontal or overhanging that it can be tilted slightly. If “horizontal” only means that a direction of the component is more horizontal than “vertical”, it does not mean that the structure must be completely horizontal, but can be tilted slightly.

In the description of the present invention, it should be noted that, unless otherwise specified and limited, the terms “arrange”, “mount”, “connected” and “connection” should be understood in a broad sense, for example, they may be fixed connection, may be detachable connection or integrated connection, may be mechanical connection, may also be electrical connection, may be direction connection, may also be indirect connection through an intermediate medium, and may be communication of two elements. A person of ordinary skill in the art may understand specific meanings of the foregoing terms in the present invention based on a specific situation.

### Embodiments

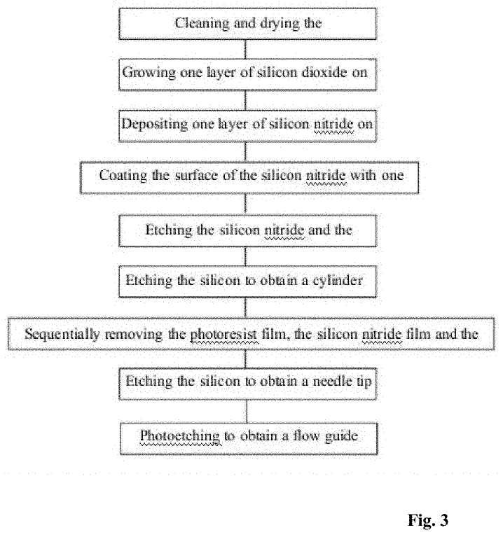

As shown in FIG. 3, the preparation method of the miniature solid silicon needle in this embodiment includes the following steps:

1) a monocrystalline silicon wafer with (100) crystal face and single side or both side polished was selected, wherein the selected monocrystalline silicon wafer is N type and has a resistivity of 5 Ω·cm (during actual operation, the resistivity ranges from 1 to 10 Ω·cm and the thickness is 700±10 μm). After being washed with standard cleaning liquid RCA1 (the molar ratio of water to ammonia water to hydrogen peroxide is 5:1:1) and RCA2 (the molar ratio of water to hydrochloric acid to hydrogen peroxide is 5:1:1), the monocrystalline silicon wafer was completely washed with deionized water and then was dehydrated and dried.

2) One layer of silicon dioxide was oxidized to grow on the monocrystalline silicon wafer according to the order of wet oxygen and dry oxygen, wherein the oxidizing temperature was controlled to below 1100° C., and the wet oxygen time and the dry oxygen time are both 5 hours.

3) One layer of silicon nitride with a thickness of 200 nm was deposited on the surface of silicon dioxide by PECVD, wherein a substrate is deposited by PECVD at the temperature of 300-450° C. and the pressure intensity of 10-270 Pa.

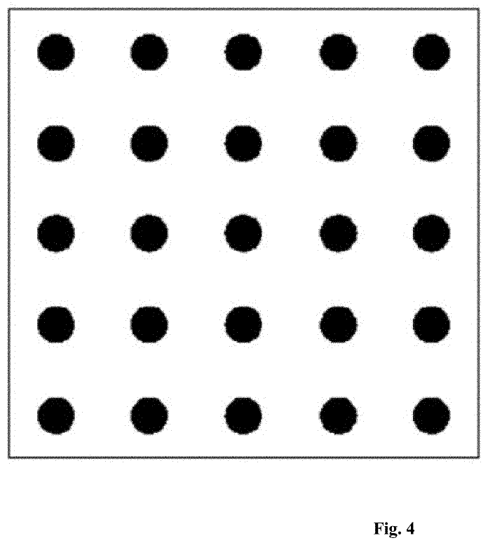

4) The silicon nitride was spin-coated with photoresist, and a pattern on a mask was transferred to the photoresist through a photoetching process, wherein the pattern of the mask is an array-shaped round spot (referring to FIG. 4), and the photoetched photoresist forms an array-shaped round shielding adhesive film. In this embodiment, an AZ9260 type photoresist is adopted, the rotating speed of spin-coating the photoresist is 2500 r/min, the time is 35 seconds, and the photoresist film has a thickness of 10 μm. Then, the monocrystalline silicon wafer was baked for 9 minutes at 95° C. After being naturally cooled, the monocrystalline silicon wafer was subjected to mask pattern photoetching, exposed for 120 seconds with a dosage of 1530 mJ/cm², developed in NMD-W developing liquid of 2.38% for 10 minutes and washed with deionized water for 1 minute, hardbaking was conducted for 15 minutes at 100° C., and the mask pattern was transferred to the photoresist, wherein the diameter of the round spot of the mask pattern is 200 μm, and a distance between circle centers of the adjacent round spots is 500 μm.

5) The silicon nitride was subjected to dry etching with a reactive ion etching machine, and after the silicon dioxide was exposed, the silicon dioxide was subjected to wet etching with mixed liquid of hydrogen fluoride and hydrofluoric acid until the monocrystalline silicon wafer was exposed. In this embodiment, the power of the reactive ion etching machine is 100 W, the gas flow of CHF3 is 100 sccm, the vacuum degree is 2.5 Pa, and the etching time is 106 minutes. Wet etching was conducted by mixed liquid of hydrogen fluoride and hydrofluoric acid (BOE liquid, 6 parts of 40 wt % hydrogen fluoride and 1 part of 49 wt % hydrofluoric acid).

Or the silicon nitride exposed outside the shielding adhesive film and the silicon dioxide located below the silicon nitride were etched by ion beams to expose the monocrystalline silicon wafer.

6) The exposed monocrystalline silicon wafer in the step 5 was subjected to anisotropic etching by an electrical coupled plasma etching system and a deep silicon etching Bosch process, and an array-shaped cylinder was etched on the monocrystalline silicon wafer. In this embodiment, the power of the electrical coupled plasma etching system is 1000 W, the etching/passivation time is 6 s/4 s as a cycle, the etching rate is 0.4 μm/cycle, and the etching resistance ratio of the monocrystalline silicon wafer to the shielding adhesive film is 42:1; and after etching for one cycle, the cylinder has a height of 160 μm and a diameter of 45 μm, and an angle of the side wall and the monocrystalline silicon wafer is 90 degrees.

7) The shielding adhesive film, the silicon nitride and the silicon dioxide at the top end of the cylinder were sequentially removed, and the top end of the cylinder was subjected to isotropic etching through KOH to form a solid micro-needlepoint cone needle and obtain a miniature solid silicon needle array chip. In this embodiment, a KOH solution of 40-80 degrees was used for etching, wherein the etching time is 30-70 minutes.

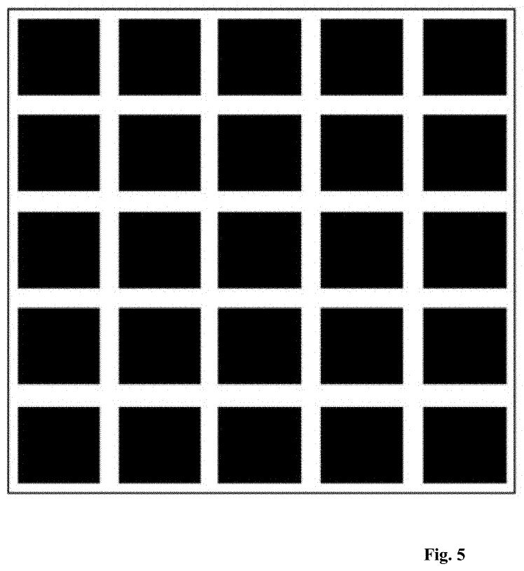

8) Then the needle body surface of the miniature solid silicon was coated with the photoresist, wherein the coating thickness of the photoresist exceeds the height of the micro-needle. The pattern on the mask was transferred to the photoresist through the photoetching process, wherein the pattern of the mask is a checkerboard-shaped block (referring to FIG. 5). The photoetched photoresist formed a block-shaped shielding adhesive film, etching was conducted, a grid-shaped flow guide groove was formed between the silicon needles, and finally, the shielding adhesive film was removed to obtain the miniature solid silicon needle array chip with the flow guide groove.

### Principle:

According to the present invention, the silicon nitride is selected as the protective film, and the etching reaction of the silicon at the bottom can be aggravated in the process of etching the silicon, so that the diameter of the base of the silicon needle is thinner.

Therefore, the present invention has the following advantages:

The exposed monocrystalline silicon wafer is subjected to anisotropic etching by a deep silicon etching Bosch process, and an array-shaped cylinder is etched on the monocrystalline silicon wafer. In the background art, a metal layer serves as a protective layer, but in the present invention, the silicon nitride serves as the protective layer, the silicon nitride can accelerate the internal etching reaction in the deep silicon etching process, so that the cylinder is thinner, and the diameter of the finally obtained needle body is thinner.

The foregoing is provided for purposes of illustrating, explaining, and describing embodiments of the present invention. Modifications and adaptations to these embodiments will be apparent to those skilled in the art and may be made without departing from the scope or spirit of the invention.

### INDUSTRIAL APPLICATION

The current invention technology is applicable to the manufacture and use of microdevices comprising microneedles, microknives and microblades, mostly for the delivery of therapeutic agents across the skin or other tissue barriers, as well as to withdraw body fluids across the skin or other tissue barriers.

What is claimed is:

1. A preparation method of a miniature solid silicon needle, comprising the following steps:
(1) selecting a single-side or double-side polished monocrystalline silicon wafer for cleaning and drying;
(2) growing one layer of silicon dioxide on the monocrystalline silicon wafer;
(3) depositing one layer of silicon nitride on the silicon dioxide;
(4) spin-coating the silicon nitride with photoresist, and transferring a pattern on a mask to the photoresist through a photoetching process, wherein the pattern on the mask is an array-shaped round spot, and the photoetched photoresist forms an array-shaped round shielding adhesive film;
(5) sequentially removing the silicon nitride located outside the shielding adhesive film and the silicon dioxide below the silicon nitride and exposing the monocrystalline silicon wafer;
(6) performing anisotropic etching on the exposed monocrystalline silicon wafer in the step 5 by utilizing an inductively coupled plasma etching system and adopting a deep silicon etching Bosch process, and etching an array-shaped cylinder on the monocrystalline silicon wafer;
(7) sequentially removing the shielding adhesive film, the silicon nitride and the silicon dioxide at a top end of the cylinder, and performing isotropic etching on the top end of the cylinder through KOH to form a solid micro-needlepoint cone needle and obtain a miniature solid silicon needle array chip; and
(8) coating a needle body surface of the miniature solid silicon needle array chip with the photoresist, transferring the pattern on the mask to the photoresist through the photoetching process, then performing etching to form a grid-shaped flow guide groove between the silicon needles, and then removing the shielding adhesive film, wherein the pattern of the mask is a checkerboard-shaped block and the photoetched photoresist forms a block-shaped shield adhesive film.

2. The preparation method of the miniature solid silicon needle according to claim 1,
wherein in the step 1, the monocrystalline silicon wafer is N type, and has a resistivity ranging from 1 to 10 Ω·cm and a thickness of 700±10 μm.

3. The preparation method of the miniature solid silicon needle according to claim 1, wherein in the step 2, one layer of silicon dioxide is oxidized to be grown on the monocrystalline silicon wafer according to the order of wet oxygen and dry oxygen, the oxidizing temperature is controlled to below 1100° C., and the wet oxygen time and the dry oxygen time are both 5 hours.

4. The preparation method of the miniature solid silicon needle according to claim 1, wherein in the step 3, a substrate is deposited by PECVD at a temperature of 300-450° C. and a pressure intensity of 10-270 Pa.

5. The preparation method of the miniature solid silicon needle according to claim 4, wherein the substrate is deposited by PECVD at a temperature of 400° C.

6. The preparation method of the miniature solid silicon needle according to claim 1, wherein in the step 3, one layer of silicon nitride with a thickness of 200 nm is deposited on a surface of the silicon dioxide by adopting PECVD.

7. The preparation method of the miniature solid silicon needle according to claim 1, wherein in the step 5, the silicon nitride exposed outside the shielding adhesive film is subjected to dry etching so that the silicon dioxide is exposed, and the silicon dioxide is subjected to wet etching.

8. The preparation method of the miniature solid silicon needle according to claim 1, wherein in the step 5, the silicon nitride exposed outside the shielding adhesive film and the silicon dioxide located below the silicon nitride may be etched by an ion beam.

9. The preparation method of the miniature solid silicon needle according to claim 7, wherein the silicon nitride is subjected to dry etching by adopting a reactive ion etching machine, and after the silicon dioxide is exposed, the silicon dioxide is subjected to wet etching with mixed liquid of hydrogen fluoride and hydrofluoric acid until the monocrystalline silicon wafer is exposed.

10. The preparation method of the miniature solid silicon needle according to claim 1, wherein in the step 7, a KOH solution of 40-80 degrees is used for etching, and an etching time is 30-70 minutes.
